(19) United States
(12) Patent Application Publication
(10) Pub. No.: US 2006/0058670 A1
(43) Pub. Date: Mar. 16, 2006

(54) Method and apparatus for ultrasound spatial compound imaging with adjustable aperture controls

(75) Inventors: Feng Lin, Waukesha, WI (US); Qian Zhang Adams, New Berlin, WI (US)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 10/915,177

(22) Filed: Aug. 10, 2004

(51) Int. Cl. A61B 8/06; A61B 8/12

(52) U.S. Cl. 600/447

(57) ABSTRACT

A method and apparatus for ultrasound spatial compounding imaging with adjustable aperture controls is disclosed. The method and apparatus can improve the image quality of all frames by applying different aperture controls on each frame of the spatially compounded image. One or both of transmit and receive aperture controls may include preventing some element of the transducer array from transmitting or receiving, calculating weighting apodizations to combine with standard apodizations for each frame, or determining an aperture size based on an f-number for the transducer array for each frame.

### BACKGROUND OF THE INVENTION

The present invention generally relates to ultrasound imaging. In particular, the present invention relates to a method and apparatus for ultrasound spatial compound imaging with adjustable aperture controls.

Spatial compounding is an advanced ultrasound imaging technique. In spatial compounding, ultrasound beams are transmitted and received in different directions. The directions may include a straight direction, as is typically performed in traditional ultrasound imagining, and steered directions that may be toward either side of the straight direction in the image plane. The image from each direction, i.e., frame, is incoherently summed together after registration to form a compounded image. The spatial compounding technique has several advantages, including: reducing speckles, enhancing boundaries, and improving contrast resolution.

However, one of the shortcomings of the technique is that the image quality of the steered frames is typically lower than the straight frame. Since steered frames are summed with the straight frame using essentially equal weighting, poor image quality in steered frames causes degradation in compounding image resolution.

The lower image quality of steered frames is partially because of the directivity of transducer elements. To characterize directivity, a directivity angle is defined. A directivity angle of an element is based at least in part on the angle between a direction perpendicular to the surface of an element and the propagation path of an ultrasound beam. A transducer element is capable of transmitting maximal acoustic pressure and receiving acoustic signal most efficiently in the direction that is perpendicular to the element's surface. This transmitting and receiving efficiency is reduced rapidly when the beam propagation path is steered. For a fixed aperture, elements at the edges may have significantly larger directivity angles for steered beams than for straight beams. Consequently, for a fixed aperture, the signal-to-noise ratio when the beam is steered is inferior to that when the beam is straight.

Grating lobe artifacts are another concern for steered frames. Grating lobes are cloud-like artifacts caused by element pitch not being smaller than half the wavelength. These artifacts are significantly worse when a beam is steered, that is, when elements have larger directivity angles.

In spatial compounding, typical practice is to apply the same transmitting and receiving apertures and apodizations on the straight frame and the steered frames. However, this is not optimal for contrast resolution. For example, an aperture setting that provides the best spatial resolution in the straight frame may result in excessive grating lobes and noise in some steered frames. On the other hand, an aperture setting that is optimal for grating lobe and noise suppression in a steered frame may result in poor spatial resolution in the straight frame.

Thus, a need exists for a method and apparatus for ultrasound spatial compounding imaging with adjustable aperture controls. Such a method and apparatus can improve the image quality of all frames by applying different aperture controls on each frame of the spatially compounded image.

### BRIEF SUMMARY OF THE INVENTION

The present invention provides a method for ultrasound spatial compound imaging with adjustable aperture controls. The method includes determining two directivity angles for an element of an ultrasound transducer array, preventing the element from transmitting and/or receiving, and combining at least the two frames to form a spatially compounded image. The two directivity angles correspond to two frames of the spatially compounded image. The element is prevented from transmitting and/or receiving for a frame if the element's directivity angle for the frame exceeds a threshold angle.

The present invention also provides a method for ultrasound spatial compound imaging using weighting apodizations. The method includes determining two directivity angles for an element of an ultrasound transducer array, calculating two ultrasound signal weighting apodizations, merging each weighting apodization with a standard apodization to create a final apodization, applying each final apodization to ultrasound signals, and combining at least two frames to form a spatially compounded image. The two directivity angles correspond to two frames of the spatially compounded image. The weighting and final apodizations also correspond to two frames of the image.

The present invention also provides a method for ultrasound spatial compound imaging with adjustable aperture controls related to f-numbers. The method includes: determining two f-numbers for a transducer array, determining two aperture sizes for the transducer array, creating at least two frames, and combining at least those two frames to form a spatially compounded image. The two f-numbers correspond to two frames of the image. The two aperture sizes correspond to two frames of the image and are based at least in part on the two f-numbers. The two frames are created by using at least the two aperture sizes.

The present invention also provides an apparatus for ultrasound spatial compound imaging with adjustable aperture controls. The apparatus includes a transducer array, an aperture directivity angle processor, an aperture element control, and a compounding processor. The transducer array includes at least one element capable of transmitting and/or receiving an ultrasound beam for one or more frames in a spatially compounded image. The aperture directivity angle processor determines a directivity angle for at least one element of the array for each of at least two frames of the image. The aperture element control prevents the element from transmitting and/or receiving the ultrasound beam for a frame if the directivity angle for that element for that frame exceeds a threshold. The compounding processor combines at least two frames to form a spatially compounded image.

The present invention also provides an apparatus for ultrasound spatial compound imaging with adjustable aperture controls using weighted apodizations. The apparatus includes a transducer array, an aperture directivity angle processor, an aperture apodization calculation processor, an aperture apodization merger processor, an aperture apodization application processor, and a compounding processor. The transducer array includes at least one element capable of transmitting and/or receiving an ultrasound beam for one or more frames in a spatially compounded image. The aperture directivity angle processor determines a directivity angle for at least one element of the array for each of at least two frames of the image. The aperture apodization calculation processor calculates two ultrasound signal weighting apodizations, each based at least in part on the respective directivity angles. The aperture apodization merger processor merges each weighting apodization with a standard signal apodization to create a final apodization for each frame. The aperture apodization application processor applies the final apodizations to ultrasound signals transmitted and/or received during at least one of the frames. The compounding processor combines at least two frames to form a spatially compounded image.

The present invention also provides an apparatus for ultrasound spatial compound imaging with adjustable aperture controls related to f-numbers. The apparatus includes a transducer array, an aperture f-number processor, an aperture size processor, and a compounding processor. The transducer array includes at least one element capable of transmitting and/or receiving an ultrasound beam for one or more frames in a spatially compounded image. The aperture f-number processor determines at least two f-numbers for the array corresponding to at least two frames of the image. The aperture size processor determines aperture sizes for the transducer array for respective frames based at least in part on the corresponding f-numbers. The compounding processor combines at least two frames to form a spatially compounded image.

### BRIEF DESCRIPTION OF THE DRAWINGS

FIG. 1 illustrates a logical component diagram of an ultrasound imaging system used in accordance with an embodiment of the present invention.

FIG. 2 illustrates the transducer of the ultrasound imaging system used in accordance with an embodiment of the present invention.

FIG. 4 illustrates a flow diagram for a method for ultrasound spatial compound imaging with adjustable aperture controls in accordance with an embodiment of the present invention.

FIG. 5 illustrates a flow diagram for a method for ultrasound spatial compound imaging with adjustable aperture controls in accordance with an embodiment of the present invention.

FIG. 6 illustrates a flow diagram for a method for ultrasound spatial compound imaging with adjustable aperture controls using weighting apodizations in accordance with an embodiment of the present invention.

FIG. 7 illustrates a flow diagram for a method for ultrasound spatial compound imaging with adjustable aperture controls related to f-numbers in accordance with an embodiment of the present invention.

FIG. 8 illustrates a logical component diagram of an ultrasound imaging system used in accordance with an embodiment of the present invention.

FIG. 9 illustrates a logical component diagram of the frame-dependent transmit aperture control used in accordance with an embodiment of the present invention.

FIG. 10 illustrates a logical component diagram of the frame-dependent receive aperture control used in accordance with an embodiment of the present invention.

FIG. 11 illustrates a logical component diagram of the frame-dependent transmit aperture control used in accordance with another embodiment of the present invention.

FIG. 12 illustrates a logical component diagram of the frame-dependent receive aperture control used in accordance with another embodiment of the present invention.

FIG. 13 illustrates a logical component diagram of the frame-dependent transmit aperture control used in accordance with another embodiment of the present invention.

FIG. 14 illustrates a logical component diagram of the frame-dependent receive aperture control used in accordance with another embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

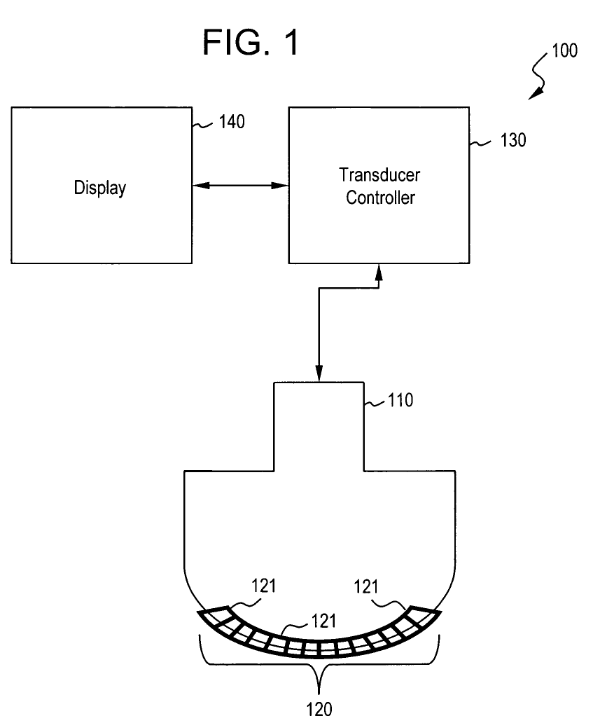

FIG. 1 illustrates a logical component diagram of an ultrasound imaging system 100 used in accordance with an embodiment of the present invention. The ultrasound imaging system 100 includes an ultrasound transducer 110, a transducer controller 130, and a display 140. The ultrasound transducer 110 includes an array 120 of transducer elements 121.

The ultrasound transducer 110 is in communication with the transducer controller 130. The transducer controller 130 is in communication with the display 140. The ultrasound transducer 110 is in communication with one or more of the elements 121 in the array 120.

The transducer controller 130 can include any processor capable of digital and/or analog communication with the transducer 110. For example, the transducer controller 130 may include a microprocessor with embedded software. As another example, the transducer controller 130 may be implemented entirely in hardware, entirely in software running on a computer or microprocessor, or some combination of hardware and software.

The transducer controller 130 may also include a computer with an input device for users of the system 100 to input imaging specifications or other information. Input imaging specifications may include one or more of a steering angle of an ultrasound beam, a focal distance or point, a frequency, a threshold angle, or an f-number, for example. For example, a user could input a steering angle of 10 degrees, a focal distance of 10 cm, a frequency of 3 MHz, a threshold angle of 30 degrees, and an f-number of 2.In addition, the transducer controller 130 may be capable of image processing.

In operation, ultrasound imaging specifications are communicated between the transducer controller 130 and the ultrasound transducer 110. The ultrasound imaging specifications can be communicated over a digital or analog signal, for example. The ultrasound imaging specifications may include one or more of a steering angle of a transmitted ultrasound beam, a focal distance, a transmit waveform, a frequency, a transmit indicator, and a receive indicator for one or more elements 121 of the array 120. A transmit indicator may include a direction to one or more elements 121 to transmit an ultrasound waveform, for example. Similarly, a receive indicator may include a direction to one or more elements 121 to receive an ultrasound waveform, for example.

In addition, an ultrasound transmission aperture size may be communicated from the transducer controller 130 to the transducer 110. Similarly, an ultrasound receive aperture size may be communicated from the transducer controller 130 to the transducer 110. The transmission and receive aperture sizes represent which elements 121 of the array 120 are to be utilized in transmitting and receiving an ultrasound beam, respectively. For example, a first aperture size may include 80% of all elements 121 of the array 120 while a second aperture size may include 60% of all elements 121 of the array 120.

Received ultrasound signals may be communicated between the transducer 110 and the transducer controller 130. Received ultrasound signals may be based on at least a strength of one or more ultrasound beams received at or measured by one or more elements 121 in the array 120.

The transducer controller 130 may also be in communication with the display 140. The received ultrasound signals from one or more of the elements 121 of the transducer array 120 can be employed by the transducer controller 130 to produce a frame of a spatially compounded image. The transducer controller 130 forms a spatially compounded image by combining two or more frames. One or more individual frames and/or a spatially compounded image may be communicated from the transducer controller 130 to the display 140.

In another embodiment of the present invention, the transducer controller 130 may be in communication with or include a data storage medium (not shown), such as a hard disk drive, tape drive, or optical drive. In this configuration, one or more individual frames and/or spatially compounded image information may be stored by the data storage medium for later display or processing.

In another embodiment of the present invention, the transducer controller 130 may be in communication with or include a network interface controller (not shown) for communication on a network, such as an Ethernet, Asynchronous Transfer Mode (ATM), or other electrical, optical, or wireless networking medium. In such an embodiment, one or more individual frames and/or spatially compounded image information may be transmitted to another device on the network for storage, processing, display, or other use.

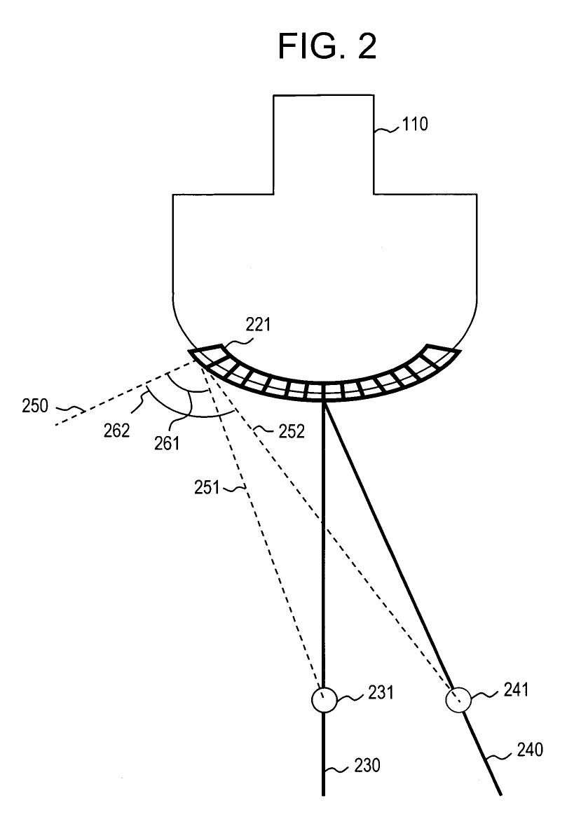

FIG. 2 illustrates the transducer 110 of the ultrasound imaging system 100 used in accordance with an embodiment of the present invention. In particular, a first element 221 is illustrated to demonstrate certain concepts. Element 221 is similar to any element 121 of the array of transducer elements 120 of the transducer 110.

In operation, the transducer 110 directs one or more of the elements 121 of the array 120 to transmit and/or receive one or more ultrasound beams. An ultrasound beam may be, for example, a straight beam 230 or a steered beam 240. A straight beam 230 can be an ultrasound beam transmitted in a direction generally along the major axis of the transducer 110. A steered beam 240 can be an ultrasound beam transmitted in a direction other than that of a straight beam 230. For example, a steered beam 240 may have a propagation path that is 10 degrees from the propagation path of a straight beam 230.

One or more elements 121 transmit one or more ultrasound beams towards a focus point. A straight beam 230 from element 221 may have a different focus point 231 than a focus point 241 for a steered beam 240, for example. Generally, a focus point 231, 241 is located at a point of interest in an ultrasound image.

A directivity angle of an element 221 can be based on at least the angle between a direction perpendicular to a transmitting or receiving surface of an element 221 and the propagation path of an ultrasound beam either transmitted or received by element 221. For example, a propagation path can include a path between the element 221 and a focal point of an ultrasound beam. For example, for a straight ultrasound beam 230 with focal point 231, the directivity angle 261 includes the angle between the direction 250 (representing a direction perpendicular to the element 221) and the path 251 between the element 221 and the focal point 231. In another example, for a steered ultrasound beam 240 with focal point 241, the directivity angle 262 includes the angle between the direction 250 and the path 252 between the element 221 and the focal point 241. The directivity angles for a single element 221 in two frames of a spatially compounded image may differ.

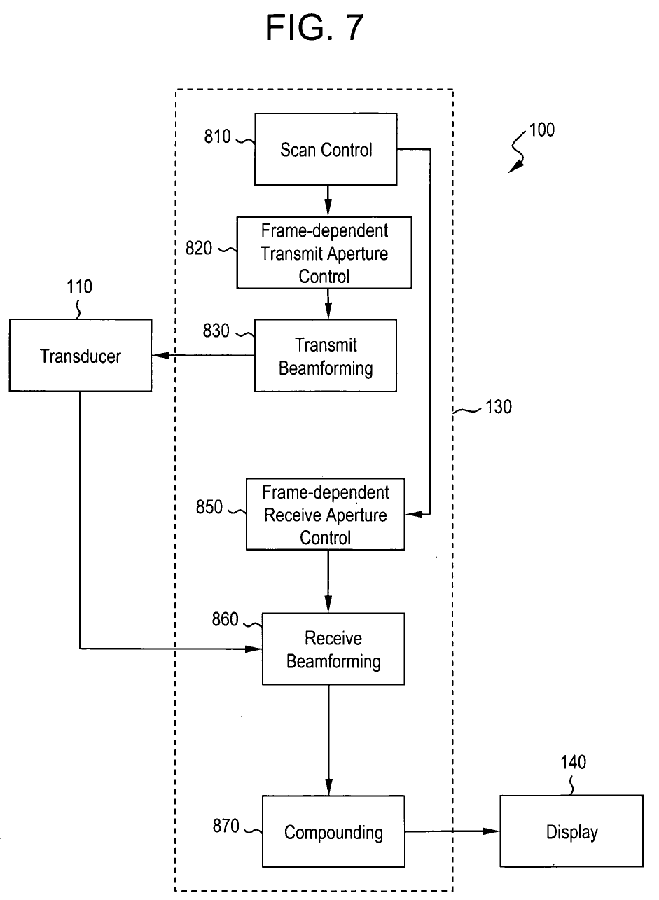

FIG. 8 illustrates a logical component diagram of an ultrasound imaging system 100 used in accordance with an embodiment of the present invention. The transducer controller 130, as exemplified in FIG. 8, includes a scan control 810, a frame-dependent transmit aperture control 820, a transmit beamforming processor 830, a frame-dependent receive aperture control 850, a receive beamforming processor 860, and a compounding processor 870.

The scan control 810 is in communication with the frame-dependent transmit aperture control 820 and the frame-dependent receive aperture control 850. The frame-dependent transmit aperture control 820 is in communication with the transmit beamforming processor 830. The transmit beamforming processor 830 is in communication with the transducer 110. The transducer 110 is in communication with the receive beamforming processor 860. The frame-dependent receive aperture control 850 is also in communication with the receive beamforming processor 860. The receive beamforming processor 860 is in communication with the compounding processor 870. The compounding processor 870 can be in communication with the display 140.

In operation, with additional reference to FIG. 2, the scan control 810 determines the directivity of one or more ultrasound beams for one or more frames of a spatially compounded image. The ultrasound beam may be, for example, a straight beam 230 or a steered beam 240. The scan control 810 may communicate ultrasound beam information to at least one of the frame-dependent transmit and receive aperture controls 820, 850. The ultrasound beam information may include, for example, the elements 121 of the transducer array 120 to be used and/or the steering angle of an ultrasound beam.

The frame-dependent transmit and receive aperture controls 820, 850 can perform various operations under one or more embodiments of the present invention, as discussed below. In general, the frame-dependent transmit and receive aperture controls 820, 850 can include of one or more processors. The aperture controls 820, 850 may provide for a different transducer 110 aperture size and/or apodization (as described below) for one or more ultrasound beams transmitted and/or received by the transducer 110. These processors may be implemented in software or hardware, and may exist as separate applications and/or devices may be integrated into one or more applications and/or devices.

The transmit beamforming processor 830 generates signals that are communicated to one or more of the elements 121 in the array 120. The signals may include, for example, a transmit aperture size of the transducer 110 and/or an ultrasound beam directivity angle for one or more of the elements 121. Based on at least these signals, the transducer 110 transmits ultrasound beams, as described above.

As described above, the transducer 110 can also receive ultrasound beams. Once the transducer 110 has received one or more ultrasound beams, the transducer 110 communicates one or more image signals to the receive beamforming processor 860. The image signals can include, for example, data based on at least one or more received ultrasound beams. The receive beamforming processor 860 can combine a plurality of the image signals to form a beam, for example.

After receiving image signals, the receive beamforming processor 860 combines a plurality of the signals to form a beam. Typically, for example, a hundred or more parallel beams may be formed. The beamforming processor 860 then communicates the beams to compounding processor 870.

The compounding processor 870 generates the spatially compounded image based on at least the beams communicated to it by the receive beamforming processor 860. The spatially compounded image may then be communicated to the display 140. The display 140 can visually display the spatially compounded image to the user.

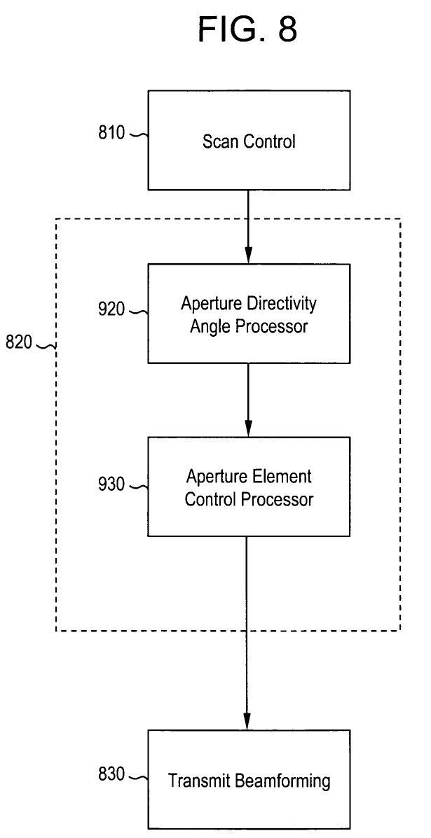

FIG. 9 illustrates a logical component diagram of the frame-dependent transmit aperture control 820 used in accordance with an embodiment of the present invention. The frame-dependent transmit aperture control 820 can include an aperture directivity angle processor 920 and an aperture element control processor 930.

The scan control 810 is in communication with the aperture directivity angle processor 920. The aperture directivity angle processor 920 is in communication with the aperture element control processor 930. The aperture element control processor 930 is in communication with the transmit beamforming processor 830.

In operation, the aperture directivity angle processor 920 calculates a directivity angle of an element, such as element 221, for a frame of a spatially compounded image. The directivity angle can be based on at least the ultrasound beam information communicated from the scan control 810, as described above. The directivity angle is communicated to the aperture element control processor 930.

The aperture control processor 930 receives the directivity angle and compares the angle to one or more threshold angles. If the aperture element control processor 930 determines that the directivity angle exceeds a threshold angle, then the element, such as 221, may be prevented from transmitting for that frame. The aperture element control processor 930 may prevent the element from transmitting by, for example, directing the transducer 110 to power down the element or to prevent the element 221 from transmitting an ultrasound beam.

The threshold angle may be specified in a variety of ways, for example, by a user input or a software protocol. In addition, the threshold angle may be determined automatically based on at least the usage of the ultrasound transducer 110. For example, the threshold angle may be determined based on at least the frequency of the ultrasound beam and/or the focal depth. A threshold angle may be, for example, 0.5 radians or 30 degrees.

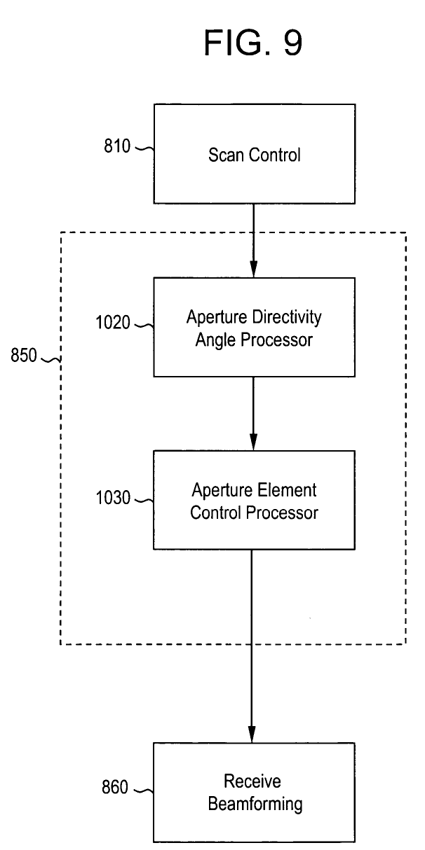

FIG. 10 illustrates a logical component diagram of the frame-dependent receive aperture control 850 used in accordance with an embodiment of the present invention. The frame-dependent receive aperture control 850 may include an aperture directivity angle processor 1020 and an aperture element control processor 1030.

The scan control 810 is in communication with the aperture directivity angle processor 1020. The aperture directivity angle processor 1020 is in communication with the aperture element control processor 1030. The aperture element control processor 1030 is in communication with the receive beamforming processor 860.

In operation, the aperture directivity angle processor 1020 calculates a receive directivity angle of an element, such as element 221, for a frame of a spatially compounded image. The receive directivity angle is communicated to the aperture element control processor 1030.

The aperture element control processor 1030 then compares the receive directivity angle to one or more threshold angles. If the aperture element control processor 1030 determines that the receive directivity angle exceeds a threshold angle, then the element, such as 221, may be prevented from receiving for that frame. The aperture element control processor 1030 may prevent the element from receiving by, for example, powering down the element or by ignoring data provided by the element.

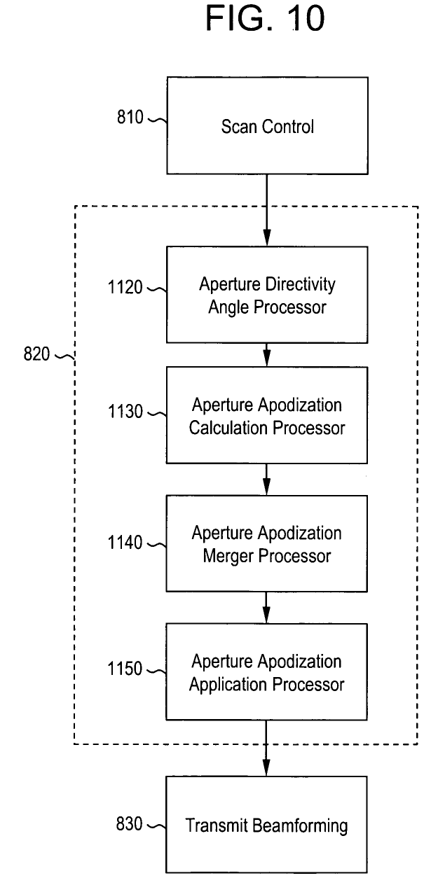

FIG. 11 illustrates a logical component diagram of the frame-dependent transmit aperture control 820 used in accordance with another embodiment of the present invention. The frame-dependent transmit aperture control 820 may include an aperture directivity processor 1120, an aperture apodization calculation processor 1130, an aperture apodization merger processor 1140, and an aperture apodization application processor 1150.

The scan control 810 is in communication with the aperture directivity angle processor 1120. The aperture directivity processor 1120 is in communication with the aperture apodization calculation processor 1130. The aperture apodization calculation processor 1130 is in communication with the apodization merger processor 1140. The aperture apodization merger processor 1140 is in communication with the aperture apodization application processor 1150. The aperture apodization application processor 1150 is in communication with the transmit beamforming processor 830.

In operation, the aperture directivity processor 1120 calculates a directivity angle of an element, such as element 221, for a frame of a spatially compounded image. The directivity angle is communicated to the aperture apodization calculation processor 1130.

The aperture apodization calculation processor 1130 calculates a weighting apodization for the transmitted ultrasound signal. The weighting apodization can be based on at least the directivity angle communicated from the aperture directivity processor 1120. The aperture apodization calculation processor 1130 communicates the weighting apodization to the aperture apodization merger processor 1140.

The aperture apodization merger processor 1140 can combine the weighting apodization received from the aperture apodization calculation processor 1130 with a standard apodization to create a final apodization. The standard apodization can include an apodization window typically used in transmit and receive apertures. Standard apodizations can have different graphical shapes, such as Gaussian, flat, or Hamming. The final apodization may also be a combination or merger of a Gaussian apodization and an apodization based on an acceptance angle, for example. The final apodization may be asymmetric. The aperture apodization merger processor 1140 communicates the final apodization to the aperture apodization application processor 1150.

The aperture apodization application processor 1150 applies the final apodization to the transmitted ultrasound signal, which is communicated to the transmit beamforming 830. Before an apodization is applied, each element in an aperture can be applied with a waveform with the same amplitude. After an apodization is applied, the waveform amplitudes can be different for elements in the aperture. Typically, the amplitude and/or weighting are largest at the center of the aperture and smallest at the aperture edges.

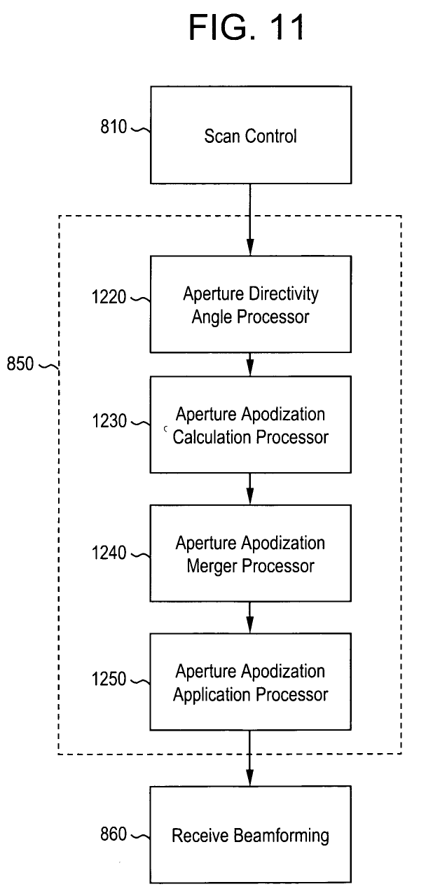

FIG. 12 illustrates a logical component diagram of the frame-dependent receive aperture control 850 used in accordance with another embodiment of the present invention. The frame-dependent receive aperture control 850 may include an aperture directivity processor 1220, an aperture apodization calculation processor 1230, an aperture apodization merger processor 1240, and an aperture apodization application processor 1250.

The scan control 810 is in communication with the aperture directivity angle processor 1220. The aperture directivity processor 1220 is in communication with the aperture apodization calculation processor 1230. The aperture apodization calculation processor 1230 is in communication with the apodization merger processor 1240. The aperture apodization merger processor 1240 is in communication with the aperture apodization application processor 1250. The aperture apodization application processor 1250 is in communication with the receive beamforming processor 860.

In operation, the aperture directivity processor 1220 calculates a receive directivity angle of an element, such as element 221, for a frame of a spatially compounded image. The receive directivity angle is communicated to the aperture apodization calculation processor 1230.

The aperture apodization calculation processor 1230 calculates a weighting apodization for the received ultrasound signal. The weighting apodization can be based, at least in part, on the directivity angle communicated from the aperture directivity processor 1220. The aperture apodization calculation processor 1230 communicates the weighting apodization to the aperture apodization merger processor 1240.

The aperture apodization merger processor 1240 merges the weighting apodization received from the aperture apodization calculation processor 1230 with a standard apodization to create a final apodization, similar to as described above. The standard apodization may be a Gaussian apodization. The final apodization may be asymmetric. The aperture apodization merger processor 1240 communicates the final apodization to the aperture apodization application processor 1250.

The aperture apodization application processor 1250 applies the final apodization to the received ultrasound signal, similar to as described above. A frame of a spatially compounded image is based on at least the application of the final apodization to the received ultrasound signal. The frame is then communicated to the receive beamforming processor 860.

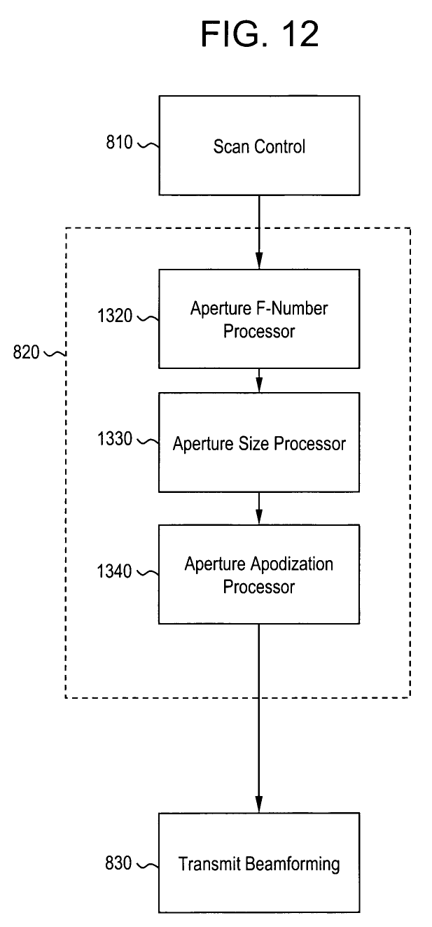

FIG. 13 illustrates a logical component diagram of the frame-dependent transmit aperture control 820 used in accordance with another embodiment of the present invention. The frame-dependent transmit aperture control 820 may include an aperture f-number processor 1320 and an aperture size processor 1330. An aperture apodization processor 1340 may also be present.

The scan control 810 is in communication with the aperture f-number processor 1320. The aperture f-number processor 1320 is in communication with the aperture size processor 1330. The aperture size processor 1330 may be in communication with the aperture apodization processor 1340. The aperture size processor 1330 may be in communication with the transmit beamforming processor 830. The aperture apodization processor 1340 may be in communication with the transmit beamforming processor 830.

In operation, the aperture f-number processor 1320 determines an f-number for the array 120 of the ultrasound transducer 110 for a frame of a spatially compounded image. The f-number can include a ratio of focal depth to aperture size. The f-number may be based on at least a threshold acceptance angle and a steering angle for an ultrasound beam for the frame. The aperture f-number processor 1320 communicates the f-number to the aperture size processor 1330.

The aperture size processor 1330 determines the aperture size of the array 120 of the ultrasound transducer based on at least the f-number. The aperture size relates to the number of elements 121 of the array 120 that are used to transmit an ultrasound beam. The aperture size processor 1330 may prevent an element from transmitting by communicating transmit indicators to the element based on whether the element is within the aperture size. The aperture size may be based on at least a focal depth for an ultrasound beam.

The aperture apodization processor 1340 applies a standard apodization to a transmitted ultrasound signal. The standard apodization may be, for example, a Gaussian apodization or a simple flat apodization. Based on at least the apodization, the transmit waveform with a proper amplitude can be applied to each element in the aperture.

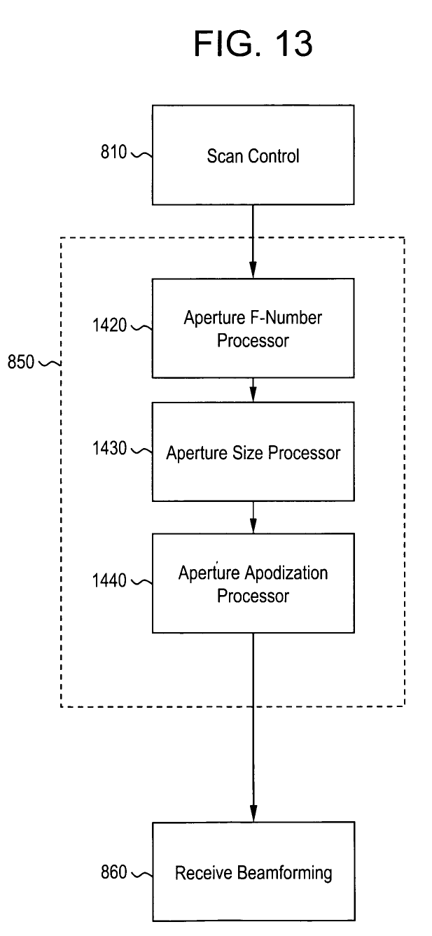

FIG. 14 illustrates a logical component diagram of the frame-dependent receive aperture control 850 used in accordance with another embodiment of the present invention. The frame-dependent receive aperture control 850 may include an aperture f-number processor 1420 and an aperture size processor 1430. An aperture apodization processor 1440 may also be present.

The scan control 810 is in communication with the aperture f-number processor 1420. The aperture f-number processor 1420 is in communication with the aperture size processor 1430. The aperture size processor 1430 may be in communication with the aperture apodization processor 1440. The aperture size processor 1430 may be in communication with the receive beamforming processor 860. The aperture apodization processor 1440 may be in communication with the receive beamforming processor 860.

In operation, the aperture f-number processor 1420 determines an f-number for the array 120 of the ultrasound transducer 110 for a frame of a spatially compounded image. The aperture f-number processor communicates 1420 the f-number to the aperture size processor 1430.

The aperture size processor 1430 determines the aperture size of the array 120 of the ultrasound transducer based on at least the f-number. The aperture size relates to the number of elements 121 of the array 120 that are used to receive an ultrasound beam. The aperture size processor 1430 may prevent an element from receiving by communicating receive indicators to the element based on whether the element is within the aperture size. The aperture size may be based on at least a focal depth for an ultrasound beam.

The aperture apodization processor 1340 applies a standard apodization to a transmitted ultrasound signal. The standard apodization may be, for example, a Gaussian apodization or a simple flat apodization. Based on the apodization, the transmit waveform with a proper amplitude is applied to each element in the aperture.

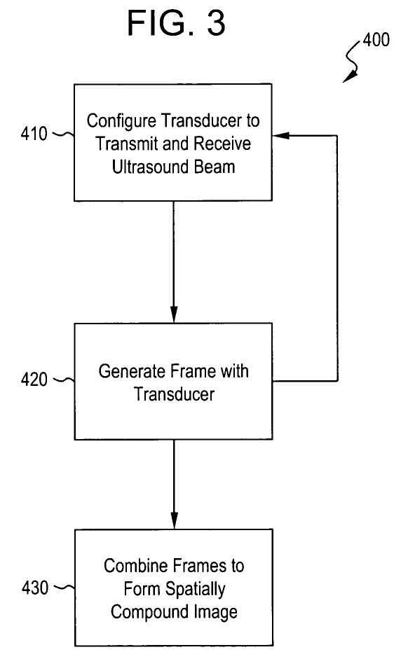

FIG. 4 illustrates a flow diagram for a method 400 for ultrasound spatial compound imaging with adjustable aperture controls in accordance with an embodiment of the present invention. The method 400 includes a step 410 of configuring the transducer to transmit and receive an ultrasound beam, a step 420 of generating a frame using the transducer, and a step 430 of combining frames to form a spatially compounded image, as described above.

In one embodiment of the present invention, step 410 is performed first, followed by step 420. These two steps are repeated at least once to produce at least two frames. Then step 430 combines at least two frames to form a spatially compounded image. Steps 410 and 420 may be performed in different ways in accordance with the present invention, as described below.

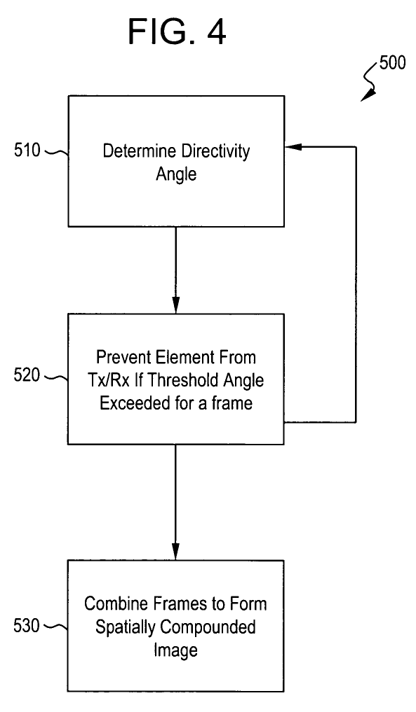

FIG. 5 illustrates a flow diagram for a method 500 for ultrasound spatial compound imaging with adjustable aperture controls in accordance with an embodiment of the present invention. The method 500 includes a step 510 of determining a directivity angle, a step 520 of preventing an element from transmitting and/or receiving for a frame, and a step 530 of combining frames to form a spatially compounded image, as described above.

In one embodiment of the present invention, step 510 is performed first, followed by step 520. These steps can be repeated at least one more time to produce at least two frames. Then, step 530 combines at least two frames to form a spatially compounded image.

In step 510, the directivity angle for at least one element of the transducer array for a given frame of a spatially compounded image is determined. For example, for element 221 of the array 120 of the ultrasound transducer 110, a directivity angle including 261 or 262 may be determined.

In step 520, the element is prevented from transmitting or receiving if the directivity angle for that element for that frame exceeds a threshold angle. For example, the element 221 of the array 120 may be prevented from one or both of transmitting and receiving if the directivity angle, for example, 262, exceeds a threshold angle. The element 221 may be prevent from transmitting by, for example, powering down the element or not allowing a signal to be communicated to the element. The element 221 may be prevented from receiving by, for example, powering down the element or by ignoring data provided by the element.

In step 530, two or more frames can be combined to form a spatially compounded image. For example, the compounding processor 870 may combine two or more frames received from the receive beamforming processor 860 to form a spatially compounded image.

By determining the directivity angle for each element for each frame, and preventing those elements that exceed the threshold angle from transmitting or receiving, the image quality of all frames can be increased. This can further result in improved contrast resolution for the spatially compounded image.

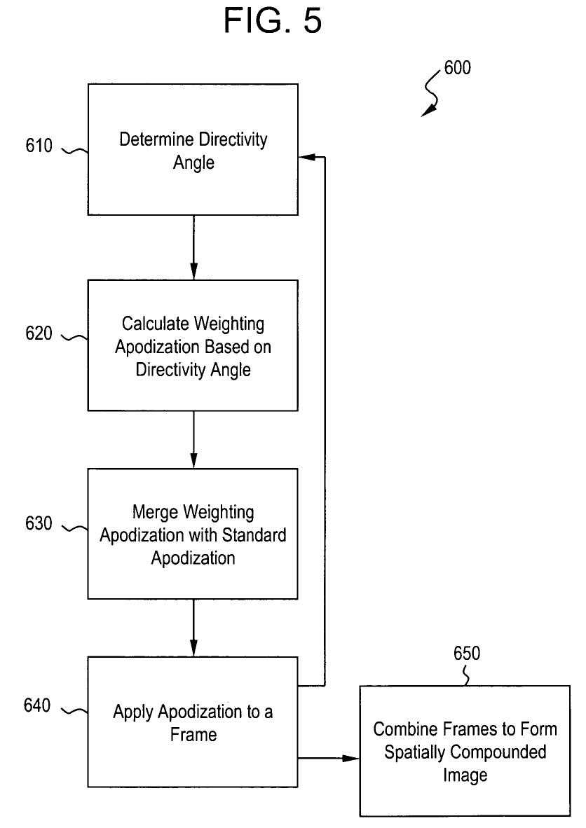

FIG. 6 illustrates a flow diagram for a method 600 for ultrasound spatial compound imaging with adjustable aperture controls using weighting apodizations in accordance with an embodiment of the present invention. The method 600 includes a step 610 of determining a directivity angle, a step 620 of calculating a weighting apodization based on at least a directivity angle, a step 630 of merging a weighting apodization with a standard apodization, a step 640 of applying an apodization to a frame, and a step 650 of combining frames to form a spatially compounded image.

In one embodiment of the present invention, step 610 is performed first, followed by step 620, next step 630, and then step 640. These steps can be repeated at least one more time to produce at least two frames. Then, step 650 combines at least two frames to form a spatially compounded image.

In step 610, the directivity angle for at least one element of the transducer array for a given frame of a spatially compounded image is determined. For example, for element 221 of the array 120 of the ultrasound transducer 110, a directivity angle including 261 or 262 may be determined.

In step 620, a weighting apodization is calculated based on a directivity angle. For example, a directivity angle including 261, 262 may be used to calculate a weighting apodization. As another example, the directivity angle may be one calculated by step 610.

In step 630, a weighting apodization is merged with a standard apodization to create a final apodization. For example, the weighting apodization calculated in step 620 may be merged with a standard apodization.

In step 640, a final apodization is applied to a frame. For example, a final apodization created in step 630 may be applied to a frame.

In step 650, two or more frames can be combined to form a spatially compounded image. For example, the compounding processor 870 may combine two or more frames received from the receive beamforming processor 860 to form a spatially compounded image.

By applying a final apodization to each frame, the image quality of all frames can be increased. This can result in improved contrast resolution for the spatially compounded image.

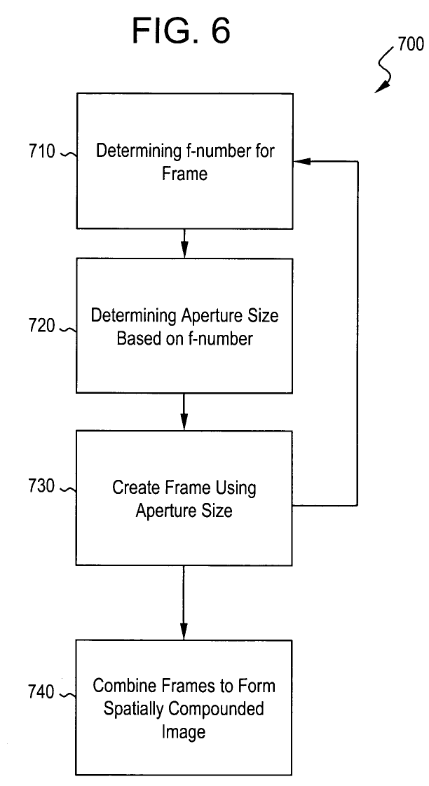

FIG. 7 illustrates a flow diagram for a method 700 for ultrasound spatial compound imaging with adjustable aperture controls related to f-numbers in accordance with an embodiment of the present invention. The method 700 includes a step 710 of determining an f-number for a frame, a step 720 of determining an aperture sized based on at least an f-number, a step 730 of creating a frame using an aperture size, and a step 740 of combining frames to form a spatially compounded image.

In one embodiment of the present invention, step 710 is performed first, followed by step 720, and then step 730. These steps are then repeated at least one more time to produce at least two frames. Then, step 740 combines at least two frames to form a spatially compounded image.

In step 710, the f-number for a given frame of a spatially compounded image is determined. A user employing method 700, for example, may determine the f-number. A user may determine the f-number based on image quality factors such as resolution, uniformity, or the presence of grating lobe artifacts. The f-number may also be based on at least a threshold acceptance angle. For example, the f-number can be large enough so that a majority of directivity angles for the various elements are smaller than the threshold acceptance angle.

In step 720, an aperture size is determined based on an f-number. For example, the aperture size may be based on an f-number determined in step 710.

In step 730, ultrasound beams are transmitted and received using the aperture size to form a frame of a spatially compounded image. The aperture size may be based, at least in part, on a focal depth for an ultrasound beam. For example, a frame of a spatially compounded image may be created using one or more aperture sizes determined in step 720. A standard apodization may also be applied to the frame created in this step.

In step 740, two or more frames can be combined to form a spatially compounded image. For example, the compounding processor 870 may combine two or more frames received from the receive beamforming processor 860 to form a spatially compounded image.

By determining an f-number and aperture size for each frame, the image quality of all frames can be increased. This can further result in improved contrast resolution for the spatially compounded image.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed is:

1. A method for ultrasound spatial compound imaging with adjustable aperture controls, said method including:
determining first and second directivity angles of a transducer array element, said first and second angles corresponding to first and second frames of a spatially compounded image, respectively;
preventing said element from at least one of transmitting and receiving an ultrasound beam for at least one of said first frame when said first directivity angle exceeds a threshold angle and said second frame when said second directivity angle exceeds said threshold angle; and
combining at least said first and second frames to form said spatially compounded image.

2. The method of claim 1, wherein said first directivity angle includes an angle between a first propagation path of said beam and a direction perpendicular to a surface of said element and said second directivity angle includes an angle between a second propagation path of said beam and said direction.

3. The method of claim 1, wherein said surface is at least one of a transmission and receiving surface of said element.

4. The method of claim 2, wherein said first and second directivity angles differ.

5. The method of claim 1, wherein said threshold angle is based on at least one or more of a transmit and receive frequency of said beam.

6. A method for ultrasound spatial compound imaging with adjustable aperture controls using weighting apodizations, said method including:
determining first and second directivity angles of a transducer array element, said first and second angles corresponding to first and second frames of a spatially compounded image, respectively;
calculating first and second ultrasound signal weighting apodizations, said first weighting apodization based on at least said first directivity angle, said second weighting apodization based on at least said second directivity angle;
merging said first weighting apodization with a standard signal apodization to create a first final apodization and said second weighting apodization with said standard signal apodization to create a second final apodization;
applying said first and second final apodizations to ultrasound signals based on at least ultrasound beams at least one of transmitted and received during said first and second frames, respectively; and
combining at least said first and second frames to form said spatially compounded image.

7. The method of claim 6, wherein at least one of said first and second directivity angles includes an angle between a propagation path of said beam and a direction perpendicular to said element.

8. The method of claim 6, wherein at least one of said first and second final apodizations is asymmetric.

9. A method for ultrasound spatial compound imaging with adjustable aperture controls related to f-numbers, said method including:
determining first and second f-numbers of a transducer array, said first and second f-numbers corresponding to first and second frames of a spatially compounded image, respectively;
determining first and second aperture sizes of said transducer array for said first and second frames, respectively, said first and second aperture sizes based on at least one or more of said first and second f-numbers;
creating said first and second frames using said first and second aperture sizes, respectively; and
combining at least said first and second frames to form said spatially compounded image.

10. The method of claim 9, wherein at least one of said first and second f-numbers include a ratio of focal depth to aperture size.

11. The method of claim 9, further including applying a standard apodization to at least one of said first and second frames.

12. The method of claim 9, wherein at least one of said first and second f-numbers are based on at least a threshold acceptance angle and a steering angle for an ultrasound beam.

13. The method of claim 12, wherein said threshold acceptance angle is based on at least one or more of a transmit and receive frequency of said ultrasound beam.

14. The method of claim 12, wherein said steering angle is based on at least a user selection.

15. The method of claim 9, wherein said first and second aperture sizes are based on at least a focal depth for an ultrasound beam.

16. An apparatus for ultrasound spatial compounding imaging with adjustable aperture controls, said apparatus including:
a transducer array including at least one element, said element capable of at least one of transmitting and receiving an ultrasound beam for at least one of first and second frames in a spatially compounded image;
an aperture directivity angle processor determining a first directivity angle of said element for said first frame and a second directivity angle of said element for said second frame;
an aperture element control preventing said element from at least one of transmitting and receiving said ultrasound beam for at least one of said first frame when said first directivity angle exceeds a threshold and said second frame when said second directivity angle exceeds said threshold; and
a compounding processor combining at least said first and second frames to form a spatially compounded image.

17. The apparatus of claim 16, wherein said first directivity angle includes an angle between a first propagation path of said beam and a direction perpendicular to a surface of said element and said second directivity angle includes an angle between a second propagation path of said beam and said direction.

18. The apparatus of claim 17, wherein said surface is at least one of a transmission and receiving surface of said element.

19. The apparatus of claim 18, wherein said first and second directivity angles differ.

20. The apparatus of claim 16, wherein said threshold angle is based on at least one or more of a transmit and receive frequency of said beam.

21. An apparatus for ultrasound spatial compounding imaging with adjustable aperture controls using weighting apodizations, said apparatus including:
a transducer array including at least one element capable of transmitting and receiving an ultrasound beam for at least one of first and second frames in a spatially compounded image;
an aperture directivity processor determining a first directivity angle of said element for said first frame and a second directivity angle of said element for said second frame;
an aperture apodization calculation processor calculating first and second ultrasound signal weighting apodizations, said first weighting apodization based on at least said first directivity angle, said second weighting apodization based on at least said second directivity angle;
an aperture apodization merger processor merging said first weighting apodization with a standard signal apodization to create a first final apodization and said second weighting apodization with said standard signal apodization to create a second final apodization;
an aperture apodization application processor applying said first and second final apodizations to ultrasound signals based on at least ultrasound beams at least one of transmitted and received during said first and second frames, respectively; and
a compounding processor combining at least said first and second frames to form a spatially compounded image.

22. The apparatus of claim 21, wherein at least one of said first and second directivity angles includes an angle between a propagation path of said beam and a direction perpendicular to a surface of said element.

23. The apparatus of claim 22, wherein said first and second propagation paths differ.

24. The apparatus of claim 21, wherein at least one of said first and second final apodizations is asymmetric.

25. An apparatus for ultrasound spatial compounding imaging with adjustable aperture controls related to f-numbers, said apparatus including:
a transducer array including at least one element, said element capable of at least one of transmitting and receiving an ultrasound beam for at least one of first and second frames in a spatially compounded image;
an aperture f-number processor determining first and second f-numbers of said array, said first and second f-numbers corresponding to said first and second frames;
an aperture size processor determining first and second aperture sizes of said transducer array for said first and second frames, respectively, said first and second aperture sizes based on at least said first and second f-numbers; and
a compounding processor combining at least said first and second frames to form a spatially compounded image.

26. The apparatus of claim 25, wherein at least one of said first and second f-numbers include a ratio of focal depth to aperture size.

27. The apparatus of claim 25, further including an aperture apodization processor, said aperture apodization processor applying a standard apodization to at least one of said first and second frames.

28. The apparatus of claim 25, wherein at least one of said first and second f-numbers are based on at least a threshold acceptance angle and a steering angle for an ultrasound beam.

29. The apparatus of claim 28, wherein said threshold acceptance angle is based on at least one or more of a transmit and receive frequency of said ultrasound beam.

30. The apparatus of claim 28, wherein said steering angle is based on at least a user selection.

31. The apparatus of claim 25, wherein said first and second aperture sizes are based on at least a focal depth for an ultrasound beam.
